(19) United States
(12) Patent Application Publication
(10) Pub. No.: US 2025/0275719 A1
(43) Pub. Date: Sep. 4, 2025

(54) METHOD OF AND SYSTEM FOR DETERMINING AND CUSTOMIZING MEDICATION DELIVERY BASED ON MEDICATION SENSITIVITY

(71) Applicant: DEKA Products Limited Partnership, Manchester, NH (US)

(72) Inventors: Dean KAMEN, Bedford, NH (US); Derek G. KANE, Manchester, NH (US); Samantha PINELLA, Manchester, NH (US)

(21) Appl. No.: 18/594,176

(22) Filed: Mar. 4, 2024

(51) Int. Cl. A61B 5/00; A61B 5/145

(52) U.S. Cl. CPC A61B 5/4848; A61B 5/14532; A61B 5/4839

(57) ABSTRACT

An embodiment configured according to principles of the invention of a method of determining sensitivity by a patient to a medication delivered in the patient in a delivery amount at a delivery frequency includes perturbing the delivery amount by a perturbation amount at a perturbation frequency, essentially defining a perturbation signal, measuring a patient response to the perturbation, and recovering the effect of the perturbation. An embodiment configured according to principles of the invention of a system for determining sensitivity by a patient to a medication delivered in a delivery amount at a delivery frequency includes a processor configured to be in communication with a pump and a sensor wherein the pump is responsive to the processor, the sensor is configured to transmit a signal that corresponds to a physiological parameter, and the processor is configured to execute a method including perturbing the delivery amount by a perturbation amount at a perturbation frequency, measuring a patient response, and recovering the patient's response to the perturbation.

### CROSS REFERENCE TO RELATED APPLICATION(S)

None.

### FIELD OF THE INVENTION

This application generally relates to infusion of medications and more specifically to ascertaining and tailoring infusion control based on a patient's sensitivity to the medication.

### BACKGROUND

Subcutaneous delivery of therapeutic fluids is a common therapy for addressing a variety of human ailments, such as diabetes. Diabetes is a condition whereby the body is unable to develop a sufficient amount of insulin to stimulate the uptake of glucose in the bloodstream. A treatment for diabetes involves delivery of insulin into the body in an amount sufficient to promote the necessary uptake of glucose.

Such delivery has been achieved with one or more injections by a syringe. The components and concentrations of the injections are intended to be appropriate for individual patient physiology. Because physiology is dynamic, patients have had to endure testing episodes, such as blood droplets taken from the fingers, many times per day.

The development of patch-sized fluid delivery systems, such as disclosed in U.S. Pat. No. 8,585,377, issued Nov. 19, 2013, to Kamen et al., which is incorporated by reference herein, radically changed the ability to treat such chronic diseases with much greater convenience and comfort for the patient. Typically, such devises have at least one reservoir for receiving a fluid not limited to insulin, and contain a power supply and a processor that executes a controller. The controller causes the pump to dispense the fluid in an amount and at a rate that is based on a program or algorithm.

Typically, the delivery rates at which insulin pumps release or deliver insulin in a patient are entered by the person living with diabetes or a caregiver of that person. Thus, the patient or caregiver determines/dictates the amount of insulin delivered for any given time/period of time. The amount typically includes a “basal” or normal state rate/amount and may include a “bolus” or enhanced rate/amount as needed for managing events such as ingesting a meal. The rate/amount is determined by the patient/caregiver based on available information or factors, such as blood glucose readings determined using a blood glucose meter, past data from like situations, intended or completed consumption, intended or completed exercise and/or stress or illness.

Although the patient determines the rate/amount based on one or more of these or additional factors, managing diabetes is not an exact science. Many reasons exist for this, not limited to inaccurate methods of delivering insulin, inaccurate blood glucose meters, inability to correctly count carbohydrate intake, inability to determine approaching illness, inability to predict the exact effects of exercise, and the inability to anticipate or forecast the effect of many additional hormones or processes in the body.

The nature of managing diabetes is complicated further by and all the more critical in view of the risk of hypoglycemia that may cause convulsions/seizures, delirium, fainting, loss of consciousness and even death. Thus, over-calculating the amount of insulin required may be life-threatening. While short-term effects of hyperglycemia are not fatal, complications due to long-term hyperglycemia are known and include shorter life span, increased risk of heart attack or stroke, kidney failure, adult blindness, nerve damage and non-traumatic amputations. Thus, under-calculating the amount of insulin required may, in the long-term, substantially affect quality of life as well as lead to fatal complications.

Development of a continuous analyte sensor that evaluates glucosamine levels in the blood greatly improved the ability to treat diabetes with greater convenience and comfort for the patient. These sensors also have created the potential for developing a closed-loop system wherein a processor in the infusion pump is informed by sensor data to configure the and instruct the pump to deliver an appropriate amount of medication according to an algorithm. Such a closed-loop system would revolutionize diabetes care through improved glycemic control with reduced monitoring requirements.

Some control algorithms are restricted to operating within a small range of variation. Others are reactive with varying ability to adjust to glucose level dynamics, such as proposed in US Patent Application Publication 2021/0241876, by Eli Lilly and Co., published Aug. 5, 2021. Unfortunately, these reactive systems lack the ability to ascertain and then pro-actively factor patient sensitivity into determining dosage amounts and frequencies that are effective for treating the disease, avoid long-term issues caused by inappropriate dosages, and improve the lives of people who must manage diabetes.

What is needed is a way to ascertain a patient's sensitivity, that is, the time lag before a patient's physiology responds to an infused medication, and the extent that physiology responds to the medication, that can be factored into an algorithm for controlling an infusion pump, thereby defining a closed-loop system whereby the patient can go about life with minimal attention to the infusion system.

The foregoing background is intended to provide a contextual overview of some current issues and is not intended to be exhaustive.

### SUMMARY OF THE INVENTION

The invention overcomes the inadequacies of existing reactive systems with a method of and system for ascertaining patient sensitivity to medication, including the time for responding and the extent of responsiveness to a given amount, that is factored into infusion control that are effective for closed-loop treatment.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. An embodiment configured according to principles of the invention of a method of determining sensitivity by a patient to a medication delivered in the patient in a delivery amount at a delivery frequency includes perturbing the delivery amount by a perturbation amount at a perturbation frequency, essentially defining a perturbation signal. The embodiment also includes measuring a patient response to the perturbation. The embodiment also includes recovering the effect of the perturbation, which defines the patient's sensitivity. Other embodiments of the invention include corresponding computer systems, apparatus, and computer programs recorded on one or more computer storage devices, each configured to perform the actions of the methods.

An embodiment configured according to principles of the invention of a system for determining sensitivity by a patient to a medication delivered in a delivery amount at a delivery frequency includes a processor configured to be in communication with a pump and a sensor. The pump is responsive to the processor. The sensor is configured to transmit a signal that corresponds to a physiological parameter. The processor is configured to execute a method including perturbing the delivery amount by a perturbation amount at a perturbation frequency, measuring a patient response, and recovering the patient's response to the perturbation.

The details of one or more embodiments are set forth in the accompanying drawings and the description below. Other features and advantages will become apparent from the description, the drawings, and the claims.

### BRIEF DESCRIPTION OF THE DRAWINGS

Non-limiting and non-exhaustive aspects of the subject disclosure are described with reference to the following figures, wherein like reference numerals refer to like parts throughout the various views unless otherwise specified.

FIG. 1 is an environmental perspective view of an embodiment of a system configured according to principles of the invention;

FIGS. 2 and 3 are diagrammatic views of a model that informs an embodiment of a method configured according to principles of the invention;

FIG. 4 is a diagrammatic view of step and wave functions that inform an embodiment of a method configured according to principles of the invention;

FIG. 5 is a diagrammatic view of model of FIGS. 2 and 3 as influenced by a method configured according to principles of the invention;

FIG. 6 are formulae referenced in the specification;

FIG. 7 is a flow diagram of a method configured according to principles of the invention;

FIGS. 8A and 8B are graphical views over time respectively of an insulin level and a glucose level;

FIGS. 9A-9C are graphical views over time respectively of a perturbation amount configured for FSK processing, a perturbed insulin level and a perturbed glucose level;

FIGS. 10A and 10B are graphical views over time respectively of demodulated high-and low-frequency glucose levels, and time-shifted demodulated low-frequency glucose levels;

FIGS. 11A and 11B are graphical views over time respectively of an insulin level and a glucose level;

FIGS. 12A-12C are graphical views over time respectively of a perturbation amount configured for heterodyne processing, a perturbed insulin level and a perturbed glucose level;

FIGS. 13A and 13B are graphical views over time respectively of demodulated high-and low-frequency glucose levels, and time-shifted demodulated high-and low-frequency glucose levels;

FIGS. 14A and 14B are graphical views over time respectively of an insulin level and a glucose level;

FIGS. 15A-15C are graphical views over time respectively of a perturbation amount configured for Wiener filtering, a perturbed insulin level and a perturbed glucose level;

FIGS. 16A and 16B are graphical views over frequency respectively of overlaid Wiener-filtered glucose and insulin signals, and time-shifted Wiener-filtered glucose levels; and

FIG. 17 is a diagrammatic view of an embodiment of a control configured according to principles of the invention.

### DETAILED DESCRIPTION OF THE PREFERRED EMBODIMENTS

The examples shown in drawings are presented to demonstrate examples of the disclosure. The drawings are illustrative and non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g., “a”, “an”, or “the”, this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B. Furthermore, to the extent that the terms “includes”, “has”, “possesses”, and the like are used in the present description and claims, such terms are intended to be inclusive in a manner similar to the term “comprising,” as “comprising” is interpreted when employed as a transitional word in a claim.

Furthermore, the terms “first”, “second”, “third”, and the like, whether used in the description or in the claims, are provided to distinguish between similar elements and not necessarily to describe a sequential or chronological order. It is to be understood that the terms so used are interchangeable under appropriate circumstances (unless clearly disclosed otherwise) and that the aspects of the disclosure described herein are capable of operation in other sequences and/or arrangements than are described or illustrated herein.

In the following description, numerous specific details are set forth to provide a thorough understanding of various aspects and arrangements. It will be recognized, however, that the techniques described herein can be practiced without one or more of the specific details, or with other methods, components, materials, etc. In other instances, well known structures, materials, or operations may not be shown or described in detail to avoid obscuring certain aspects.

Reference throughout this specification to “an aspect,” “an arrangement,” “a configuration,” or “an example” indicates that a particular feature, structure, or characteristic is described. Thus, appearances of phrases such as “in one aspect,” “in one arrangement,” “in a configuration,” “in some examples,” or the like in various places throughout this specification do not necessarily each refer to the same aspect, feature, configuration, example, or arrangement. Furthermore, the particular features, structures, and/or characteristics described may be combined in any suitable manner.

To the extent used in the present disclosure and claims, the terms “component,” “system,” “platform,” “layer,” “selector,” “interface,” and the like are intended to refer to a computer-related entity or an entity related to an operational apparatus with one or more specific functionalities, wherein the entity may be either hardware, a combination of hardware and software, software, or software in execution. As an example, a component may be, but is not limited to being, a process running on a processor, a processor, an object, an executable, a thread of execution, a program, and/or a computer. By way of illustration and not limitation, both an application running on a server and the server itself can be a component. One or more components may reside within a process and/or thread of execution and a component may be localized on one computer and/or distributed between two or more computers. In addition, components may execute from various computer-readable media, device-readable storage devices, or machine-readable media having various data structures stored thereon. The components may communicate via local and/or remote processes such as in accordance with a signal having one or more data packets (e.g., data from one component interacting with another component in a local system, a distributed system, and/or across a network such as the Internet with other systems via the signal). As another example, a component can be an apparatus with specific functionality provided by mechanical parts operated by electric or electronic circuitry, which may be operated by a software or firmware application executed by a processor, wherein the processor can be internal or external to the apparatus and executes at least a part of the software or firmware application. As yet another example, a component can be an apparatus that provides specific functionality through electronic components without mechanical parts; the electronic components can include a processor therein to execute software or firmware that confers at least in part the functionality of the electronic components.

To the extent used in the subject specification, terms such as “store,” “storage,” “data store,” data storage,” “database,” and the like refer to memory components, entities embodied in a memory, or components comprising a memory. It will be appreciated that the memory components described herein can be either volatile memory or nonvolatile memory, or can include both volatile and nonvolatile memory.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject disclosure and claims should generally be construed to mean “one or more” unless specified otherwise or clear from context to be directed to a singular form.

The words “exemplary” and/or “demonstrative,” to the extent used herein, mean serving as an example, instance, or illustration. For the avoidance of doubt, the subject matter disclosed herein is not limited by disclosed examples. In addition, any aspect or design described herein as “exemplary” and/or “demonstrative” is not necessarily to be construed as preferred or advantageous over other aspects or designs, nor is it meant to preclude equivalent exemplary structures and techniques. Furthermore, to the extent that the terms “includes,” “has,” “contains,” and other similar words are used in either the detailed description or the claims, such terms are intended to be inclusive, in a manner similar to the term “comprising” as an open transition word, without precluding any additional or other elements.

As used herein, the term “infer” or “inference” refers generally to the process of reasoning about, or inferring states of, the system, environment, user, and/or intent from a set of observations as captured via events and/or data. Captured data and events can include user data, device data, environment data, data from sensors, application data, implicit data, explicit data, etc. Inference can be employed to identify a specific context or action or can generate a probability distribution over states of interest based on a consideration of data and events, for example.

The disclosed subject matter can be implemented as a method, apparatus, or article of manufacture using standard programming and/or engineering techniques to produce software, firmware, hardware, or any combination thereof to control a computer to implement the disclosed subject matter. The term “article of manufacture,” to the extent used herein, is intended to encompass a computer program accessible from any computer-readable device, machine-readable device, computer-readable carrier, computer-readable media, or machine-readable media. For example, computer-readable media can include, but are not limited to, a magnetic storage device, e.g., hard disk; floppy disk; magnetic strip(s); an optical disk (e.g., compact disk (CD), digital video disc (DVD), Blu-ray Disc (BD)); a smart card; a flash memory device (e.g., card, stick, key drive); a virtual device that emulates a storage device; and/or any combination of the above computer-readable media.

Generally, program modules include routines, programs, components, data structures, etc., that perform particular tasks or implement particular abstract data types. The illustrated aspects of the subject disclosure may be practiced in distributed computing environments where certain tasks are performed by remote processing devices that are linked through a communications network. In a distributed computing environment, program modules can be located in both local and remote memory storage devices.

Computing devices can include at least computer-readable storage media, machine-readable storage media, and/or communications media. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, compact disk read only memory (CD ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media that can be used to store desired information. In this regard, the terms “tangible” or “non-transitory” herein as applied to storage, memory, or computer-readable media, are to be understood to exclude only propagating transitory signals per se as modifiers, and do not exclude any standard storage, memory, or computer-readable media that are not only propagating transitory signals per se.

Computer-readable storage media can be accessed by one or more local or remote computing devices, e.g., via access requests, queries, or other data retrieval protocols, for a variety of operations with respect to the information stored by the medium.

A system bus, as may be used herein, can be any of several types of bus structure that can further interconnect to a memory bus (with or without a memory controller), a peripheral bus, and a local bus using any of a variety of commercially available bus architectures. A database, as may be used herein, can include basic input/output system (BIOS) that can be stored in a non-volatile memory such as ROM, EPROM, or EEPROM, with BIOS containing the basic routines that help to transfer information between elements within a computer, such as during startup. RAM can also include a high-speed RAM such as static RAM for caching data.

As used herein, a computer can operate in a networked environment using logical connections via wired and/or wireless communications to one or more remote computers. The remote computer(s) can be a workstation, server, router, personal computer, portable computer, microprocessor-based entertainment appliance, peer device, or other common network node. Logical connections depicted herein may include wired/wireless connectivity to a local area network (LAN) and/or larger networks, e.g., a wide area network (WAN). Such LAN and WAN networking environments are commonplace in offices and companies, and facilitate enterprise-wide computer networks, such as intranets, any of which can connect to a global communications network, e.g., the Internet.

When used in a LAN networking environment, a computer can be connected to the LAN through a wired and/or wireless communication network interface or adapter. The adapter can facilitate wired or wireless communication to the LAN, which can also include a wireless access point (AP) disposed thereon for communicating with the adapter in a wireless mode.

When used in a WAN networking environment, a computer can include a modem or can be connected to a communications server on the WAN via other means for establishing communications over the WAN, such as by way of the Internet. The modem, which can be internal or external, and a wired or wireless device, can be connected to a system bus via an input device interface. In a networked environment, program modules depicted herein relative to a computer or portions thereof can be stored in a remote memory/storage device.

When used in either a LAN or WAN networking environment, a computer can access cloud storage systems or other network-based storage systems in addition to, or in place of, external storage devices. Generally, a connection between a computer and a cloud storage system can be established over a LAN or a WAN, e.g., via an adapter or a modem, respectively. Upon connecting a computer to an associated cloud storage system, an external storage interface can, with the aid of the adapter and/or modem, manage storage provided by the cloud storage system as it would other types of external storage. For instance, the external storage interface can be configured to provide access to cloud storage sources as if those sources were physically connected to the computer.

As employed in the subject specification, the term “processor” can refer to substantially any computing processing unit or device comprising, but not limited to comprising, single-core processors; single-core processors with software multithread execution capability; multi-core processors; multi-core processors with software multithread execution capability; multi-core processors with hardware multithread technology; vector processors; pipeline processors; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a state machine, a discrete gate or transistor logic, discrete hardware components, or any combination thereof designed to perform the functions described herein. Processors can exploit nano-scale architectures such as, but not limited to, molecular and quantum-dot based transistors, switches, and gates, in order to optimize space usage or enhance performance of user equipment. A processor may also be implemented as a combination of computing processing units. For example, a processor may be implemented as one or more processors together, tightly coupled, loosely coupled, or remotely located from each other. Multiple processing chips or multiple devices may share the performance of one or more functions described herein, and similarly, storage may be effected across a plurality of devices. A processor may be implemented to reside in a cloud-based network such as, e.g., the Internet.

The actions of a method or algorithm described in connection with the arrangements disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other known form of storage medium. A storage medium may be coupled to the processor such that the processor can read information from, and write information to, the storage medium. In the alternative, the storage medium may be integral to the processor. The processor and the storage medium may reside in an ASIC. The ASIC may reside in functional equipment such as, e.g., a computer, a robot, a user terminal, a mobile telephone or tablet, a car, or an IP camera. In the alternative, the processor and the storage medium may reside as discrete components in such functional equipment. Additionally or in the alternative, at least one of the processor and/or the storage medium may reside in a cloud-based network such as, e.g., the Internet.

Configurations of the present teachings are directed to computer systems for accomplishing the methods discussed in the description herein, and to computer readable media containing programs for accomplishing these methods. The raw data and results can be stored for future retrieval and processing, printed, displayed, transferred to another computer, and/or transferred elsewhere. Communications links can be wired or wireless, for example, using cellular communication systems, military communications systems, and satellite communications systems. Parts of the system can operate on a computer having a variable number of CPUs. Other alternative computer platforms can be used.

The present configuration is also directed to software/firmware/hardware for accomplishing the methods discussed herein, and computer readable media storing software for accomplishing these methods. The various modules described herein can be accomplished on the same CPU, or can be accomplished on different CPUs. In compliance with the statute, the present configuration has been described in language more or less specific as to structural and methodical features. It is to be understood, however, that the present configuration is not limited to the specific features shown and described, since the means herein disclosed comprise preferred forms of putting the present configuration into effect.

Methods can be, in whole or in part, implemented electronically. Signals representing actions taken by elements of the system and other disclosed configurations can travel over at least one live communications network. Control and data information can be electronically executed and stored on at least one computer-readable medium. The system can be implemented to execute on at least one computer node in at least one live communications network. Common forms of at least one computer-readable medium can include, for example, but not be limited to, a floppy disk, a flexible disk, a hard disk, magnetic tape, or any other magnetic medium, a compact disk read only memory or any other optical medium, punched cards, paper tape, or any other physical medium with patterns of holes, a random access memory, a programmable read only memory, and erasable programmable read only memory (EPROM), a Flash EPROM, or any other memory chip or cartridge, or any other medium from which a computer can read. Further, the at least one computer readable medium can contain graphs in any form, subject to appropriate licenses where necessary, including, but not limited to, Graphic Interchange Format (GIF), Joint Photographic Experts Group (JPEG), Portable Network Graphics (PNG), Scalable Vector Graphics (SVG), and Tagged Image File Format (TIFF).

Various arrangements are described herein. For simplicity of explanation, the methods or algorithms are depicted and described as a series of steps or actions. It is to be understood and appreciated that the various arrangements are not limited by the actions illustrated and/or by the order of actions. For example, actions can occur in various orders and/or concurrently, and with other actions not presented or described herein. Furthermore, not all illustrated actions may be required to implement the methods. In addition, the methods could alternatively be represented as a series of interrelated states via a state diagram or events. Additionally, the methods described hereafter are capable of being stored on an article of manufacture, as defined herein, to facilitate transporting and transferring such methodologies to computers.

The invention is a method of determining a patient's sensitivity to a medication and then adjusting medication delivery parameters based on that sensitivity. Sensitivity is discerned from the patient's responsiveness to precisely-timed perturbations of delivery amounts.

Although insulin and diabetes are discussed herein, the invention is not limited to use of the systems and methods for treating only diabetes. The disclosed methods and systems may be used for the delivery of any fluid, including any medical or therapeutic fluid, including but not limited to, insulin, for the treatment of a medical condition not limited to diabetes mellitus.

Described herein are methods and systems for closed loop or partially closed loop control of diabetes. As described above, many factors affect the amount of insulin a patient or user requires to maintain an appropriate blood glucose level. The term “appropriate” is used herein to mean a blood glucose level which has been chosen by the patient and/or their health-care provider as healthy for the patient. The appropriate blood glucose level for each patient may vary, as will the appropriate blood glucose level at any given time for any given patient. In general, many health-care providers recommend maintaining blood glucose levels between 90-140 mm/dl. However, depending on the circumstance, the range may vary. For example, a patient may deem a blood glucose level of 150 mg/dl appropriate before bedtime, but would consider the same reading inappropriate before mealtime.

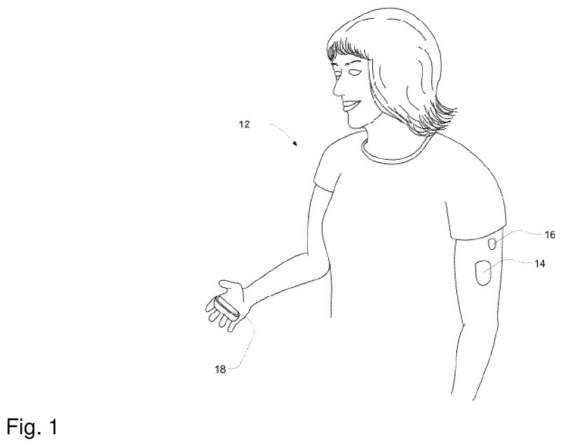

Referring to FIG. 1, a preferred system configured according to principles of the invention includes a patient 12 wearing a medical fluid pump 14, a sensor apparatus 16 and holding a controller 18. The sensor apparatus 16 may contain one or more continuous glucose monitor (“CGM”) and one or more additional sensors. The sensors transmit data to the controller 18. The medical fluid pump 14 is shown as a patch pump similar to any one of the patch pumps shown and described in U.S. Published Application No. US-2007-0219480, published Sep. 20, 2007 and entitled Patch-Sized Fluid Delivery Systems and Methods (E72); U.S. Published Application No. US-2007-0228071, published Oct. 4, 2007 and entitled Fluid Delivery Systems and Methods (E70); U.S. Published Application No. US-2007-0219496, published Sep. 20, 2007 and entitled Pumping Fluid Delivery Systems and Methods Using Force Application Assembly (E71); U.S. Published Application No. US-2007-0219597, published Sep. 20, 2007 and entitled Adhesive and Peripheral Systems and Methods for Medical Devices (E73); U.S. patent application Ser. No. 12/347,985, filed Dec. 31, 2008 and entitled Infusion Pump Assembly (G75); U.S. patent application Ser. No. 12/347,982, filed Dec. 31, 2008 and entitled Wearable Pump Assembly (G76); U.S. patent application Ser. No. 12/347,981, filed Dec. 31, 2008 and entitled Infusion Pump Assembly (G77); U.S. patent application Ser. No. 12/347,984, filed Dec. 31, 2008 and entitled Pump Assembly With Switch (G79); U.S. Published Application No. US-2009-0099522, published Apr. 16, 2009 and entitled Microneedle Systems and Apparatus (G34); and U.S. Published Application No. US-2009-0099523, published Apr. 16, 2009 and entitled Infusion Pump Assembly (G46), which are hereby incorporated herein by reference in their entirety.

Preferably, pump 14 is controlled by and transmits information to the controller 18. Some embodiments may include a user interface allowing for control by the patient/user. Controller 18 receives information relating to the one or more sensors and the pump. The controller additionally receives inputs from the user, e.g., events, and may receive manual inputs for fingerstick readings or fingerstick data. Additionally, the controller, in some embodiments, may receive information relating to food or glucose readings, etc., wirelessly. In some embodiments, the controller includes voice recognition, thus, in these embodiments, the controller may receive commands via voice.

The system may use at least one CGM. CGMs include a glucose sensor (referred to as a “sensor” or “analyte sensor”). In various embodiments, the CGM sensor is introduced and remains in the user's interstitial fluid located on the body, e.g., on the abdomen. The CGM sends electrical signals at predetermined intervals to a receiver or controller. The receiver or controller correlates these electric signals to a glucose value. In some embodiments, redundant CGMs are used to provide more than one interstitial glucose reading at any given reading time for safety concerns. In some embodiments, the redundant CGMs may be one or more additional CGMs (the same CGM) located in different parts of the patient. In other embodiments, the redundancy may be provided by one or more sensors integrated onto a single CGM apparatus where all of the sensors are introduced into a similar place on the patient and in some embodiments, using the same auto inserter. In some embodiments, one or more redundant sensors may be sensors introduced to different depths in the patient, e.g., if there are 4 redundant sensors, each sensor is introduced to a different depth in the patient.

Redundant sensors provide additional safety. The sensor readings may be sent to a processor which may use various methods to determine if the system should accept the reading, or which reading the system should accept, for use in determining the amount of insulin to deliver. For example, the processor may determine if the values vary more than 6%, for example (in other embodiments, the percentage different may be different and may be determined and/or specified based one or more calibration techniques) then the readings may not be used for delivery and re-calibration (i.e., by a finger-stick) is required. If the processor does not receive a signal from one, the processor may be programmed to ignore that sensor. If all redundant sensors read the same or similar value (again, within a percentage that may be pre-programmed or may be pre-determined), then the system may be more confident the value is closer to correct.

In some embodiments, the redundant sensors may be calibrated differently. For example, one sensor may be calibrated to be more sensitive than the other sensor(s). In some embodiments, the various sensors are tuned to different dynamic ranges. For example, where two sensors are used, each of the two sensors are tuned to a different range, one is tuned to be very sensitive to low blood glucose levels, the other tuned to high blood glucose levels. If for example, the sensor tuned low is reading 60 mg/dl, the system will recognize that the sensor is in the patient and reading. If the sensor tuned high is reading 250 mg/dl, the system may confirm the sensor is in the patient and reading. In other embodiments, the redundant sensors may be tuned based on a time-constant, i.e., one sensor reads faster than the next, etc.

The controller serves as at least one user interface, and also a central user interface for the CGM(s)/sensors, the pump, and the patient's/user's interface with the control system. For purposes herein, the controller may be programmed by a patient, a “user”, a care-giver, a health-care provider or any combination thereof. For purposes of this description however, the term “patient” or “patient/user” or “user” refers to anyone entering information into to controller, or utilizing the controller to provide care for the patient. In the exemplary embodiment, the system controller communicates with the various system components via wireless, e.g., radio frequency (“RF”) communication and/or other types of remote communication. In the exemplary embodiment, the controller includes a graphical user interface (“GUI”) and one or more input device, e.g., button(s), capacitive slider(s), jog wheel, touch screen, keypad, electronic keypad, and any other input device. The controller also includes at least one processor, although in the exemplary embodiments, the controller includes at least two processors, a control processor and a safety processor. These processors may be redundant processors, or two different processors providing redundant processing or checking the processing of one another.

Some embodiments of the controller may include at least one “event” or specialty button, e.g., a “food” button, an “exercise” button, and a “bolus” button. In some embodiments, the controller may contain a single “event” button. Pressing or actuating this button may bring the user to an event menu, which may include a list of potential events, one or more of which may be customizable to the user.

With respect to all event buttons, these buttons, when pressed, would bring the patient/user either to a menu or a processing logic that enables the patient/user to input directly into the processing logic for exercise, food or bolus, for example. The logic may then query the patient/user to enter additional information, for example, how long the exercise is expected to last, how rigorous, how much food (i.e., how may carbohydrates), glycemic index, fat content and protein content of the food. With respect to bolus, the patient/user would be able to input the volume of a bolus by using a series of button presses or by using another input device, i.e., jog wheel, button or slider, to input the requested volume of insulin, i.e., the units of insulin. In some embodiments, the user interface includes many of the same features as found on insulin pumps and pump controllers known in the art.

In the exemplary embodiment, the controller also includes a “strip reader”, e.g., a space that accepts a glucose test strip for use in “finger stick” or “fingerstick” readings, e.g., the patient pricks their fingers and uses the blood from the finger to apply to the “finger stick”. The “strip reader”, using electrochemical testing, determines the blood glucose level of the blood. The strip reader may be used to calibrate the CGM, to double check unexpected or unusual readings, or as a back-up to the CGM in case of CGM failure. In some embodiments, the strip reader may be a separate device, such as a glucose meter. In these embodiments, the glucose meter may either wirelessly receive the fingerstick reading or the user may manually input the reading into the controller.

The controller, in some embodiments, serves as the receiver for the at least one sensor, including but not limited to, the at least one CGM. As such, the user will indicate to the controller when a new sensor is introduced into the body. In some embodiments, the user may additionally input the location of the sensor on the user's body, e.g., which include, but are not limited to, right abdomen, left abdomen, right arm, left arm, right hip, left hip, right left, left leg, etc. This may be desirable as the sensor may perform differently in different areas on the body. As the controller records and processes these data, the controller may calibrate the sensor based on past profile information indicating “lag” and/or “drift” information from the same area of the body.

To manage diabetes using at least a partially closed-loop method, the components of the system described may be used to deliver controlled volumes of insulin and, in some embodiments, a counter regulatory hormone, e.g., glucagon, according to a variety of methods, some of which are described herein. In the exemplary embodiments, the control methods rely on the use of a system that includes the ability to actively measure the volume of insulin or other fluid that is actually delivered to the patient rather than measuring the volume of insulin requested by the user or pre-programmed by a user to be delivered. At least one CGM and a user interface and processes containing instructions for the at least partial closed loop algorithm. Other sensors and data input models may also be included, as described in more detail above. However, in some embodiments, pumps that do not actively measure the volume of insulin or other fluid that the pump is actually delivering to the patient may also be used. In these embodiments, an assumption is made that the volume delivered to the patient is the volume requested by the processor unless or until a mechanical malfunction or occlusion is detected.

An embodiment of a method of determining sensitivity to medication configured according to principles of the invention can be adopted for ascertaining sensitivity to any of various medications. Sensitivity refers to: (1) an amount of time that elapses before the patient responds to the medication; and (2) a response correspondence or extent that the patient responds to an amount of medication. Here, the method of determining these important attributes is described in the context of, but is not limited to insulin sensitivity.

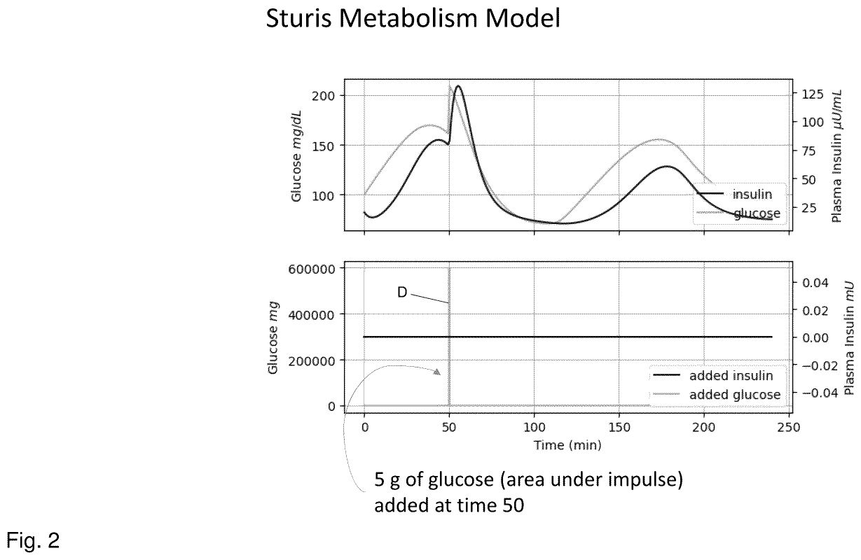

Referring to FIG. 2, an insulin-related embodiment of the invention is configured in view of the dynamic model developed to mimic human insulin-glucose metabolism known as the Sturis Metabolism model, as described in J. theor. Biol. (2000) 207, 361-375, and “Computer Model For Mechanisms Underlying Ultradian Oscillations Of Insulin And Glucose” Sturis, Polonsky, Mosekilde, Van Cauter, Am J Physiol. 1991 May; 260(5 Pt 1):E801-9. doi: 10.1152/ajpendo.1991.260.5.E801 which are incorporated by reference herein. The Sturis model has been adapted to allow for outside sources of glucose and insulin. Detailed description of the model is outside the scope of this document. Suffice it to impart that the Sturis model describes the concentrations of insulin and glucose in the body, and functions related to their increase, decrease and utilization with interrelated time-differential equations. The model includes the following feedback loops: glucose stimulates pancreatic insulin secretion, insulin stimulates glucose uptake and inhibits hepatic glucose production, and glucose enhances its own uptake. The system contains two significant delays. One delay relates to the correlation of the physiological action of insulin on the utilization of glucose with the concentration of insulin in a slowly equilibrating intercellular compartment rather than with the concentration of insulin in the plasma. The other delay is associated with the time lag between the appearance of insulin in the plasma and its inhibitory effect on hepatic glucose production.

FIG. 2 shows an expected relationship between concentrations of glucose and insulin in non-diabetic humans, with levels of glucose leading that of insulin. Introducing a dose of glucose D into the system causes a corresponding, time-delayed increase in insulin production or release. The invention provides for supplying insulin in diabetics in a way that mimics non-diabetic systems.

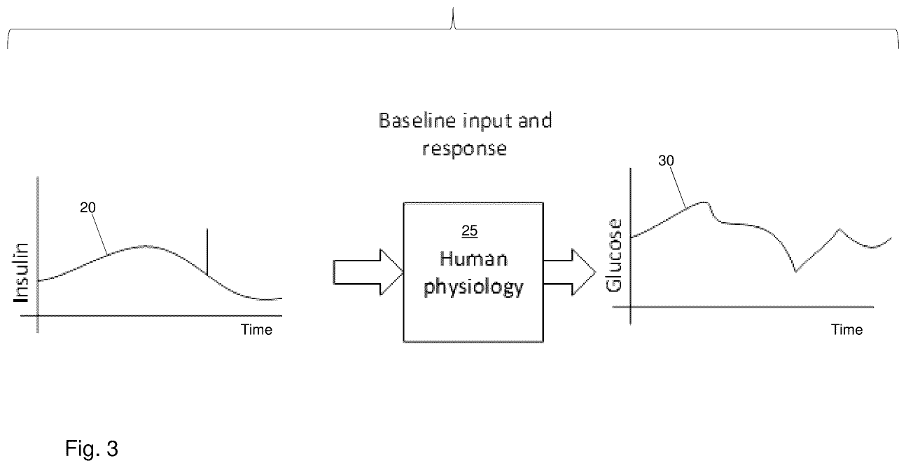

Referring to FIG. 3, consistent with the Sturis model, an amount of insulin available in the body over time, represented by curve 20, undergoes known human physiological processes 25 that reduce an amount of glucose found in the blood over time, represented by curve 30. Ordinarily, the pancreas secretes insulin into the bloodstream throughout the day and night, referred to as a basal insulin release. This basal amount is not fixed but varies according to perceived glucose concentration levels. Glucose levels rise and fall, for example, as the body develops glucose, such as from a meal, and absorbs glucose into the cells for which insulin is needed. Responsive to elevated blood glucose concentrations, the pancreas increases the amount of insulin released into the bloodstream beyond the basal amount. Typically, glucose and insulin concentrations correspond, with the former leading the latter by an amount that varies from person to person.

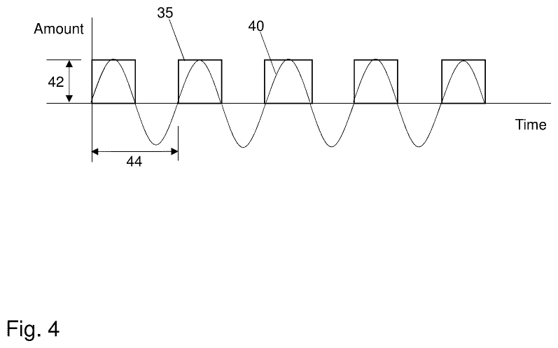

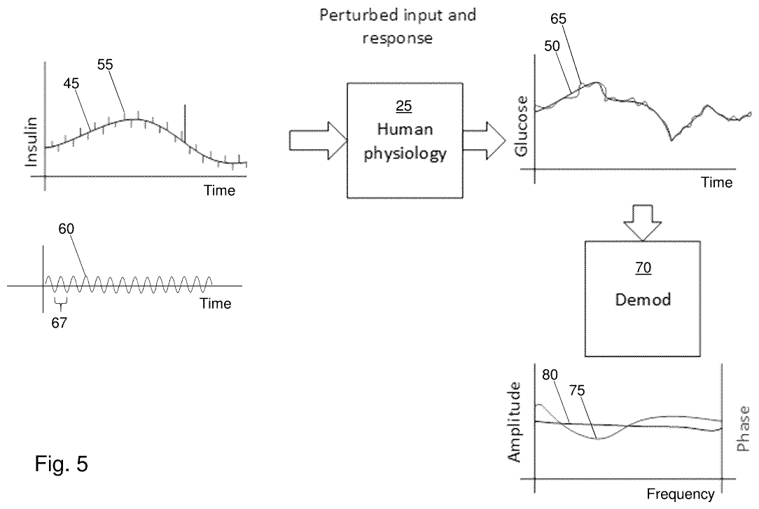

Referring to FIG. 4, when the pancreas is not functioning adequately to produce a sufficient amount of insulin to reduce glucose concentration levels, insulin supplementation typically is achieved via injections administered by a syringe and/or infusion pumps. Syringe injections introduce a singular amount of insulin at one time. Infusion pump injections typically release discrete amounts of insulin in the body at discrete times, as modeled by a stepped curve 35 or positive range of a sine curve 40 having an amplitude 42 and a period 44. Viewed over a greater stretch of time, preferably, the discrete injections of curves 35 and 40 effectively may be modeled as a smooth curve 45, as shown in FIG. 5, comparable to curve 20 in FIG. 3 corresponding to the healthy, continuous-release functioning of a human pancreas.

Patient sensitivity to insulin, that is, the extent that a patient reacts to an amount of insulin, and the lag time between when the insulin is introduced and when the body reacts thereto, is unique to each patient. Given the dire consequences of ill-timed releases or superabundant or inadequate amounts of insulin, understanding this sensitivity is paramount to patient comfort if not survival.

One approach has been, considering patient inputs, namely anticipated exercise and meal quantities, and inferring sensitivity from the difference between predicted and measured glucose levels. However, the control system's parameters must be held constant or inferred from the glucose response under control and the assumed accuracy of meal/exercise reporting. Another drawback is that control of insulin/glucose is slower and more conservative than where parameters are determined through active excitation of the physiology, such as according to principles of the invention.

### Single-Tone Perturbation

The invention improves on the former approach through active excitation and monitoring of a patient's physiology. This and succeeding embodiments of the invention employ of principles of signal processing for discerning both the extent and frequency of a patient's sensitivity to active excitation.

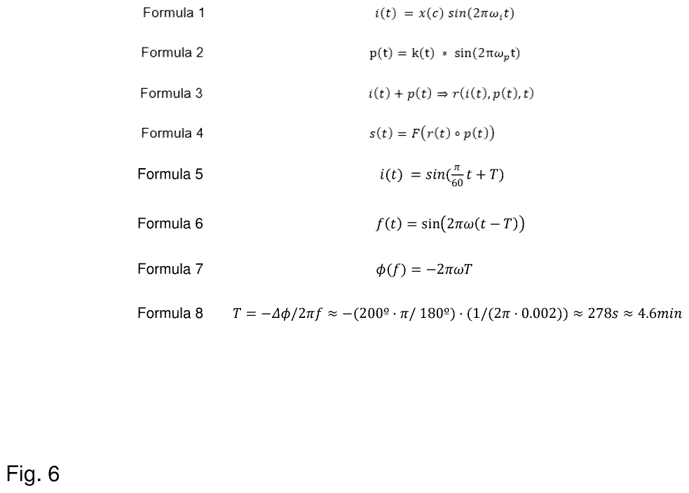

Continuing to refer to FIG. 5, the method presumes that supplemental insulin is released with an infusion pump according to an algorithm or function that controls the amounts released and the frequency at which the amounts are released and describes insulin release curve 45. Consistent with the modeling of FIG. 3, the algorithm or function for achieving insulin release curve 45 may be described according to Formula 1 below and in FIG. 6.

$$i ( t ) = x ( c ) sin ( 2 {πω}_{i} t ) \qquad Formula 1$$

where i is the amount of insulin released, t is time, x is an amount of insulin required that varies according to glucose concentration (c), and ωᵢ is the frequency at which insulin releases occur.

As with the model in FIG. 3, responsive to the presence of the insulin, known human physiological processes 25 reduce an amount of glucose found in the blood as shown in glucose level curve 50. Preferably, responsive to the one or more sensors described above, the algorithm controlling the pump responds like a human pancreas and releases insulin such that glucose and insulin concentrations correspond, with the former leading the latter.

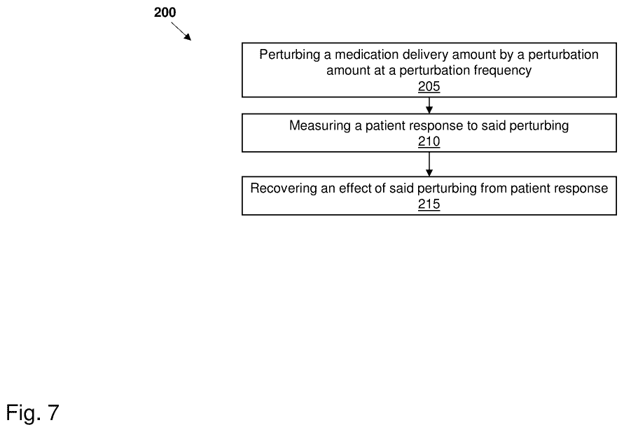

Referring also to FIG. 7, a preferred embodiment of the invention is method 200 which includes a step 205 of perturbing the delivery amount 45 by a perturbation amount 60 at a perturbation frequency 67. Preferably, but not necessarily, the invention employs a sinusoidal perturbation model that perturbs the insulin release algorithm or function and analyzes the patient's responses to the perturbations. The perturbations may be constant, as shown in curve 60, or varied to explore the extent of a patient's responsiveness, as described below. The amount that the insulin release algorithm is perturbed may be described according to Formula 2 below and in FIG. 6.

$$p ( t ) = k ( t ) * sin ( 2 {πω}_{p} t ) \qquad Formula 2$$

where p is the amount that release i(t) is increased or decreased, t is time, k is an amount of perturbation that may be constant or varied with time, and Ωₚ is the frequency at which the perturbations occur. Perturbing insulin release curve 45 by the amounts of perturbation curve 60 yields curve 45 with periodic spikes 55.

A preferred embodiment of method 200 includes a step 210 of measuring patient response. As shown in FIG. 5, a patient may respond to the perturbed infusion and exhibit a glucose level that corresponds to curve 65, a perturbed response that corresponds to the perturbed insulin release of curves 45/55. The release, perturbation and response may be expressed as Formula 3 below and in FIG. 6.

$$i ( t ) + p ( t ) ⇒ r ( {i ( t )}_{7} + p ( t ) , t ) \qquad Formula 3$$

A preferred embodiment of method 200 includes a step 215 of recovering the effect of the perturbation 75 from the perturbed patient response 65. Sensitivity is determined from the recovered effect. A preferred way to recover the effect of perturbation is by demodulating the measured glucose level with sine/cosine signals of the same frequency of the perturbation. This recovers the amplitude of the physiology's response to excitation at this frequency. Preferably, this response is determined through an interpretation function applied to the convolution of the response and perturbation signal which may be expressed by Formula 4 below and in FIG. 6.

$$s ( t ) = F ( r ( t ) ∘ p ( t ) ) \qquad Formula 4$$

In one embodiment of the invention, the perturbation signal is a sine wave, p(t)=A sin(Ωₚt), and perturbation curve 60 is understood as a carrier signal with encoded information, namely, the physiological response. A patient's unique physiology 25 responds to the insulin release and develops a glucose level that corresponds to curve 65. Removing the perturbation signal of curve 60 from the glucose response signal 65 is understood as demodulating signal 65 by signal 60. This demodulation 70 yields, in the frequency domain, a sensitivity signal 75 having amplitudes that correlate with the patient's individual responsiveness physiology.

Consistent with principles of signal processing, the frequency Ωₚ at which the perturbations occur, preferably, is less than the frequency Ωₘ at which measurements occur and/or consistent with Nyquist sampling to permit meaningful demodulation of the patient's response.

An advantage of this approach is that it rapidly recovers a patient's physiology's sensitivity to insulin. One limitation is that the measurement time lag, the period of physiological response, must be less than the perturbation period.

Once a patient's sensitivity is understood, it can be used to enhance the algorithm that is programmed to control a pump that supplies the patient with insulin. For example, if the sensitivity signal reveals that the patient takes a longer time to respond to insulin, then the sensitivity function would adjust the algorithm to time needed releases earlier in a particular cycle. For another example, if the sensitivity signal reveals that the patient responds to small amounts of insulin, then the sensitivity function would adjust the algorithm to release less amounts of insulin that the algorithm ordinarily would have for a particular cycle. Adjusting a pumping algorithm based on the patient's sensitivity flattens the patient's responsiveness curve 80, as shown in FIG. 5, which leads to more normal body functioning, greater comfort and better health.

### Frequency Shift Keying

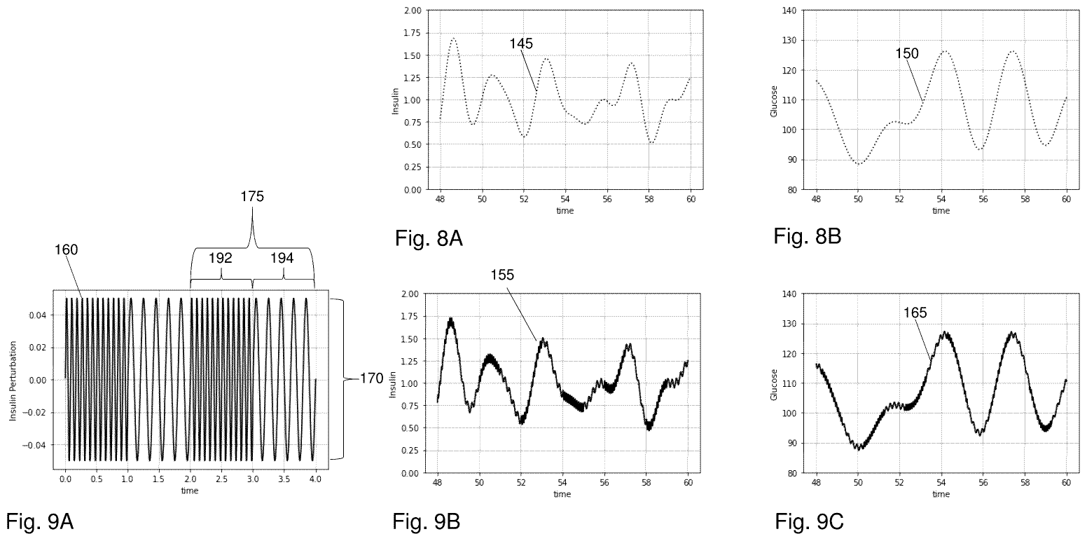

Referring to FIGS. 8A-B and 9A-C, another embodiment of a method for discerning patient sensitivity according to principles of the invention perturbs and evaluates insulin releases according to principles of frequency shift keying (FSK). The method involves perturbing the insulin delivery by an amount at a frequency, defining a single tone, and varying the amount and frequency according to a known schedule, such as after five periods. Demodulation of the physiological response yields a sensitivity curve or function.

FIGS. 8A-B represent an unperturbed state wherein the control or algorithm of a pump supplies insulin in an amount that corresponds to curve or signal 145 as shown in FIG. 8A. The patient's physiology responsive to the input insulin yields a glucose curve or signal 150 as shown in FIG. 8B.

This embodiment of a method configured according to principles of the invention perturbs the insulin release function that describes insulin release curve 145 in FIG. 8A with the perturbation function that describes the perturbation curve 160 in FIG. 9A. The perturbation signal is configured according to principles of frequency key shifting (FSK). The FSK signal 160 has a fixed amplitude or perturbation amount 170 that is added to the insulin release function within fixed periods of time have distinct frequency signals, a higher frequency 192 or faster perturbation and a lower frequency 194 or slower perturbation as shown in FIG. 9A.

Referring to FIG. 9B, adding or modulating insulin release function/curve 145 with the perturbation function/curve 160 yields perturbed insulin release curve 165. The mathematics involved in modulating the insulin release function with the perturbation function are similar to that described above with respect to the foregoing perturbation model except that, in Formula 2, Ωp, the frequency at which the perturbations occur, is varied in a known way. Consequently, the perturbed insulin release signal 155 has diverse frequency components that are physiologically recognizable and indicative of different attributes of medication sensitivity.

Referring to FIG. 9C, responsive to the perturbed insulin release signal 155, the patient's unique physiology will develop glucose levels comparable to glucose response signal/curve 165. The expectation is that the high frequency insulin signal will carry through to the glucose response 165. If the perturbation is small relative to the individual's physiology, then an essentially linear response may be expected.

The glucose response signal/curve 165 contains valuable information about the physiology, namely: insulin sensitivity, the extent that the physiology reacts to an amount of insulin; and the response time or lag between insulin input and glucose level change.

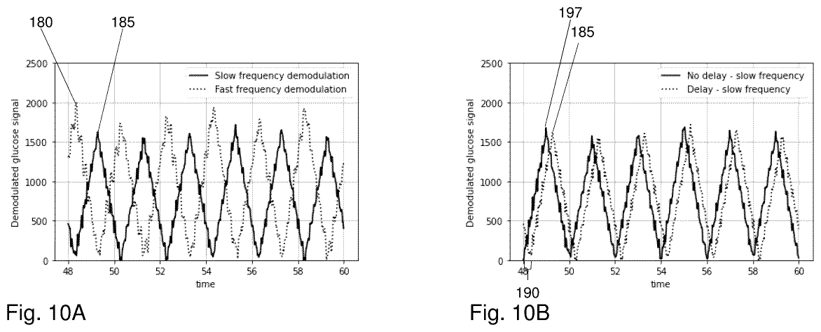

Referring to FIG. 10A, the method provides for removing the perturbation signal of curve 160 from the glucose response signal 165, demodulating signal 165 by signal 160. FSK demodulation consists of standard IQ demodulation, where I stands for the in-phase component and Q stands for the quadrature phase component, carried out at each of the component frequencies. An IQ demodulator strips data from a modulated signal by creating I and Q (amplitude and phase) components of the signal that can be interpreted in meaningful ways, in this case, medication sensitivity.

As shown in FIG. 10A, corresponding to the higher frequency portion 192 and lower frequency portion 194 of perturbed insulin release function 160, the two respective demodulation signals 180 and 185 alternate. When the demodulation 180 of the glucose response signal 165 is larger than the demodulation 185 of the slower frequency signal 194, the demodulation 180 corresponds to the fast frequency portion 192 of the FSK signal as shown in FIG. 9A and vice versa. The variation in amplitude relative to the vertical axis indicates a variation of insulin sensitivity or the extent that the physiology is reactive to insulin. The variation in sensitivity between the two frequencies can also be determined. Thus, the method may reveal that the dynamics of the patient's physiology.

Referring to FIG. 10B, the response time of the patient's physiology can be determined by evaluating the phase shift between the insulin signal and the glucose signal. For example, the slower component 194 of the FSK input signal 175 may be described in terms of a sine function such as in formula 5 below and in FIG. 6.

$$i ( t ) = sin ( \frac{π}{60} t + T ) \qquad Formula 5$$

where t is time in seconds and T is a constant. The input signal has a period of 2 minutes, thus i(0)=i(120). The demodulated response signal 185 would be similar to the input signal with a similar periodicity but shifted by an amount of time corresponding to the response time of the body's physiology. Accordingly, as shown in FIG. 10B, the lower-frequency demodulation signal 185, shown overlaid a non-time-shifted instance of the lower-frequency demodulation signal 197, has a difference 190 between the actual and time-shifted signals that corresponds to the patient's temporal responsiveness or time lag. The actual and time-shifted high-frequency demodulation signals exhibit the same information.

An advantage that multi-tone FSK demodulation has over single-tone perturbation is the ability to determine response times or lags up to the entire length 175 of the perturbation frequency signal in the FSK wave form. Assuming that the FSK wave form has a length 175 that exceeds the physiological response time, since the faster portion 192 is less than length 175, then the effect of fast frequency perturbation would be discernable from the demodulations, that effect possibly occurring within or without the period of the fast frequency perturbation.

Implementing the FSK model reveals insulin sensitivity at known frequencies, including unambiguous time lags up to the length of time spent at a frequency. An advantage of this approach is obtaining a greater signal-to-noise ratio at particular frequencies. However, the technique is slower than heterodyning, as described below.

### Code Division Multiplexing

Another embodiment of a method for discerning patient sensitivity configured according to principles of the invention perturbs and evaluates insulin releases according to principles of code division multiplexing (CDM). Similar to the FSK embodiment described above, the CDM signal includes codes that are multiple blocks long instead of just the two described above for FSK. This approach extends time-lags between perturbations/responses, which can be measured.

### Heterodyning

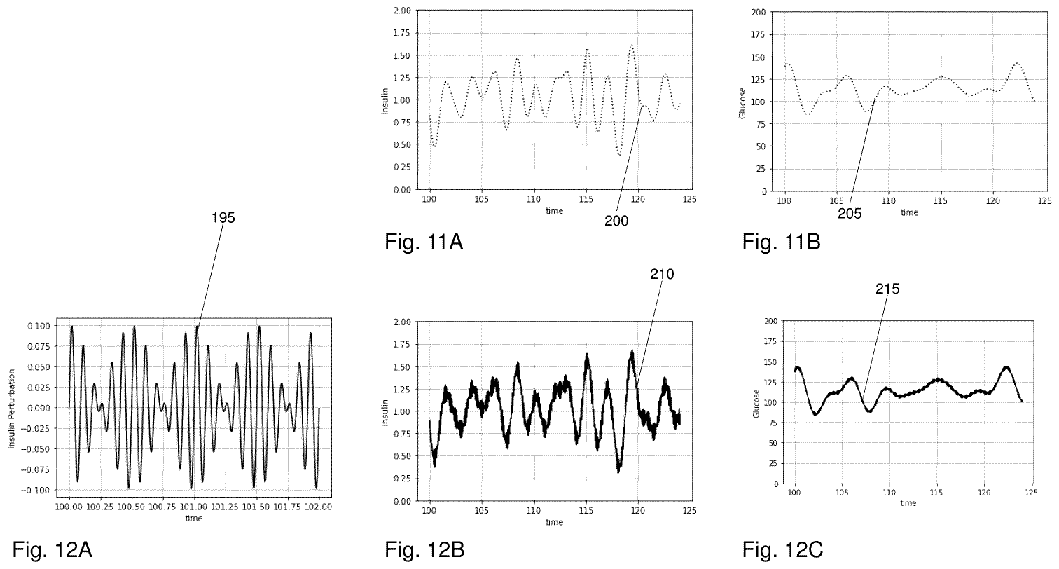

Referring to FIGS. 11A and B and 12A-C, another embodiment of a method for discerning patient sensitivity configured according to principles of the invention perturbs and evaluates insulin releases according to principles of heterodyning. This embodiment is similar to the FSK embodiment except that the perturbation signal 195 employs two or more tones continuously. In this non-limiting example only two tones are shown.

FIGS. 11A-B represent an unperturbed state wherein the control or algorithm of a pump supplies insulin in an amount that corresponds to curve or signal 200 as shown in FIG. 11A. The patient's physiology responsive to the input insulin yields a glucose curve or signal 205 as shown in FIG. 11B.

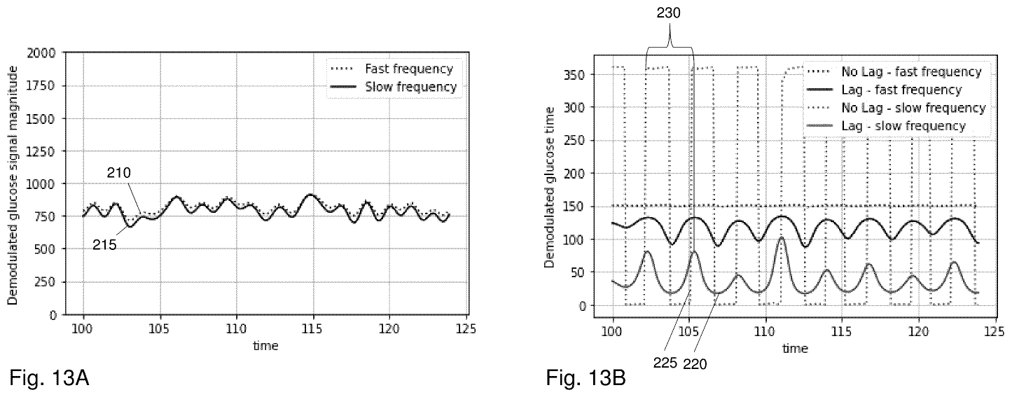

Referring to FIGS. 13A and B, adding the heterodyne signal 195 to the pump controller command signal 200 yields a heterodyned signal 210 with higher frequency components.

Referring to FIG. 12C, the patient's physiology responds to the insulin released according to the perturbed signal 210 and presents glucose levels that describe curve 215. The expectation is that the high frequency insulin signal 210 will carry through to the glucose response 215. Smaller perturbations will tend to yield more linear responses.

Referring to FIG. 13A, heterodyne demodulation consists of standard IQ demodulation carried out at each of the component frequencies. The variation in amplitude indicates a variation of insulin sensitivity. The variation in sensitivity between the two frequencies can also be determined.

Referring to FIG. 13B, the patient's physiological response time or time lag can be inferred from the time lag that is observable in either signal. The lower-frequency demodulation signal 220, shown overlaid a time-shifted instance of the lower-frequency demodulation signal 225, has a phase that is shifted by an amount 230 relative to that of the perturbation signal. The amount 230 between the actual and time-shifted signals corresponds to the patient's temporal responsiveness or time lag. The phase shifts observable in the actual and time-shifted demodulation signals at other frequencies exhibit the same information. Both of the “no lag” curves essentially are flat. The lower frequency curve toggles between close to 0 degrees and close to 360 degrees. The variation is due to noise in the signal.

In heterodyning, the phase measurements at distinct frequencies permit determining the physiological time delay. A unique smallest time lag should correspond to the time difference in both signals. For example, if the heterodyne includes a sine wave having a period of 7 minutes and a sine wave with a period of 11 minutes, and if the 7-minute sine wave has an offset of 3 minutes in glucose while the 11-minute sine wave has an offset of 5 minutes, because the offset should be the same for both, a common time should give this combination of offsets:
1) At 10 minutes the 7-minute sine wave has a 3-minute offset and the 11-minute sine wave a 10-minute offset;
2) At 17 minutes the 7-minute sine wave has a 3-minute offset and the 11-minute sine wave has a 6-minute offset;
3) At 24 minutes the 7-minute sine wave has a 3-minute offset and the 11-minute sine wave has a 2-minute offset;
4) At 31 minutes the 7-minute sine wave has a 3-minute and the 11-minute sine wave has a 9 minute offset;
5) At 38 minutes the 7-minute sine wave has a 3-minute offset and the 11-minute sine wave has a 5-minute offset

It follows that the insulin time delay is 38 minutes because it is compatible with both phase measurements.

The signal processing in heterodyning is more complex than FSK or CDM and more sensitive to other signals in the system bleeding into the demodulation. Selecting distinct enough frequencies for long time period measurements may be challenging depending on individual physiology. The time range measurable essentially is the period of the beat frequency, f_{fast}-f_{slow}.

### Wiener Filtering

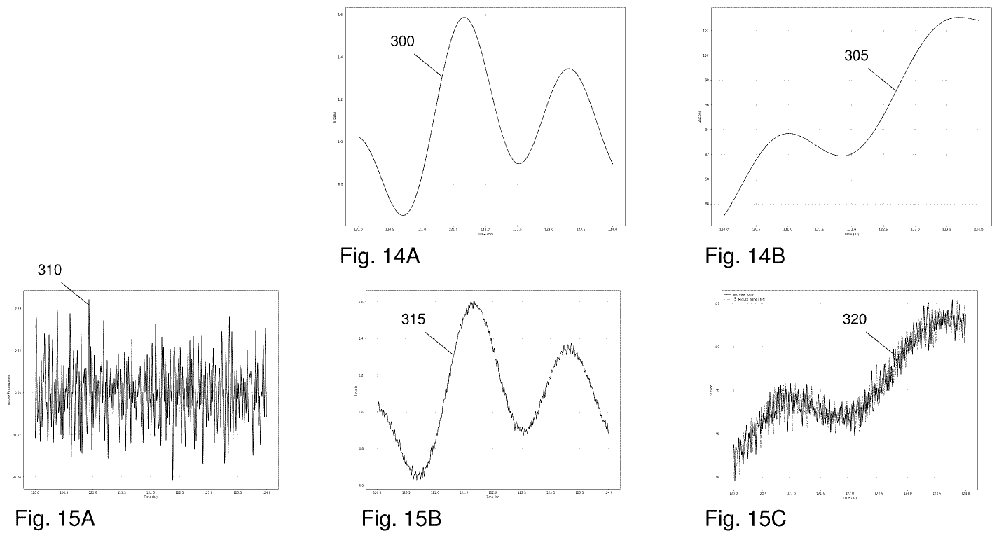

Referring to FIGS. 14A and B and 15A-C, another embodiment of a method for discerning patient sensitivity configured according to principles of the invention perturbs and evaluates insulin releases according to principles of Wiener filtering. This embodiment is similar to Heterodyning but employs a continuous band of frequencies.

FIGS. 14A-B represent an unperturbed state wherein the control or algorithm of a pump supplies insulin in an amount that corresponds to curve or signal 300 as shown in FIG. 14A. The patient's physiology responsive to the input insulin yields a glucose curve or signal 305 as shown in FIG. 14B.

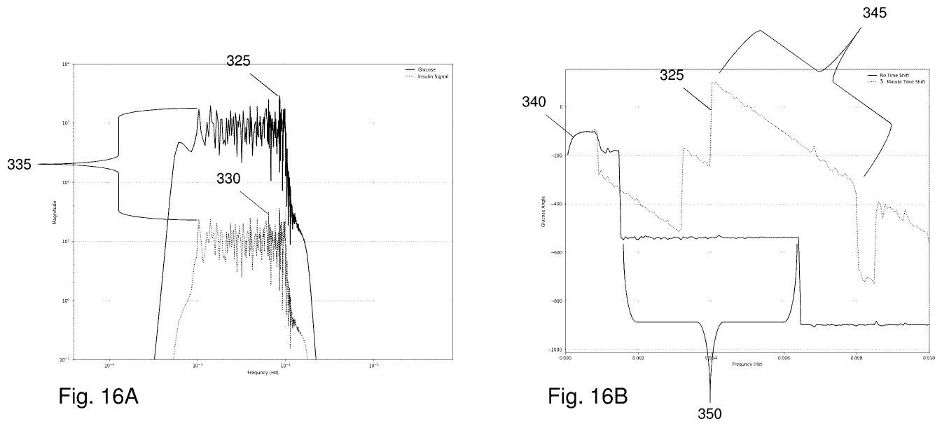

Referring to FIGS. 15A and B, preferably, the invention adds a narrow-band insulin perturbation signal 310 to the pump controller command signal 300 to achieve a signal 315 with complex frequency components.

Referring to FIG. 15C, the patient's physiology responds to the insulin released according to the perturbed signal 310 and presents glucose levels that describe curve 320. The expectation is that the variety of frequencies of the perturbation signal 310 will carry through to the glucose response 320.

Referring to FIG. 16A, the Wiener filter is applied to the response signal 320, defining a Wiener filtered response signal 325, and to the input signal 315, defining a Wiener filtered input signal 330, across all frequencies. Amplitude differences 335 between the Wiener filtered response signal 325 and the Wiener filtered input signal 330 indicate variations of insulin sensitivity depending on insulin amount.

Referring to FIG. 16B, the patient's physiological response time or time lag can be inferred from the phase shift between the output and input signals. This shift is observed by comparing a time-shifted instance 340 of the Wiener filtered response signal overlaying a non-time-shifted instance of the Wiener filtered response signal 325. The generally flat response curve, such as in section 350, indicates being in phase with the input signal, the perturbed insulin signal 315 of FIG. 15B. The relatively skewed response curve, such as in section 345, indicates being out of phase with the perturbed insulin signal 315. The phase shift between curves 340 and 325 is indicative of the patient's temporal responsiveness or time lag T. The response signal 325 with the time lag T can be described by Formula 6 below and in FIG. 6.

$$f ( t ) = sin ( 2 πω ( t - T ) ) \qquad Formula 6$$

The phase in radians is described by Formula 7 below and in FIG. 6.

$$ϕ ( f ) = - 2 πω T \qquad Formula 7$$

As shown on FIG. 16B, response signal 325 exhibits a change of about 200° at a change in frequency of 0.002 Hz. Substituting values for the variables yields Formula 8 below and in FIG. 6.

$$T = - Δϕ / 2 π f \approx - ( 200 \underline{°} \cdot π / 180 \underline{°} ) \cdot ( 1 / ( 2 π \cdot 0.002 ) ) \approx 278 s \approx 4.6 min \qquad Formula 8$$

The invention contemplates applying other signal processing techniques for discerning patient sensitivity to medications as would be appropriate. This sensitivity information improves algorithms that currently drive infusion pumps and renders them able to modify in accordance with a patient's physiology. This individually-tailored self-corrective capacity enables deploying infusion pumps as a closed-loop system with which a patient can go about daily life with minimal attention to the pump.

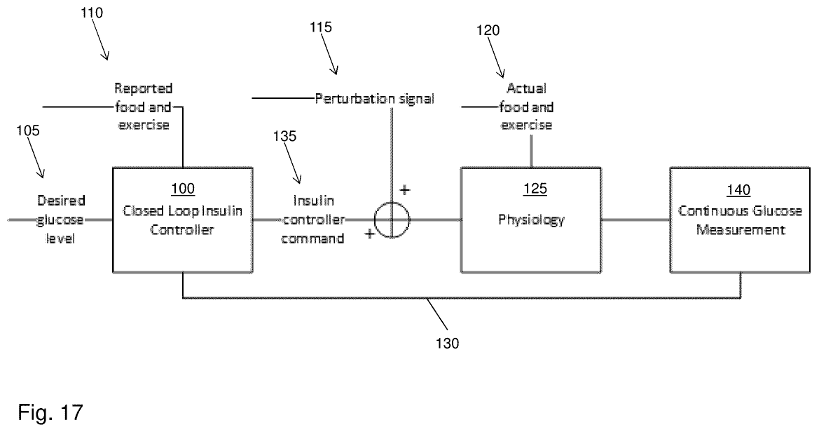

Referring to FIG. 17, an embodiment of a control 100 configured according to principles of the invention, preferably, is closed looped and permits a patient to go about daily activities without having to manage the pump. To this end, preferably, control 100 is a multiple input, multiple output (MIMO) controller. Some of the inputs to controller 100 are not limited to a desired glucose level 105; predicted or reported food intake 110; predicted or reported exercise events 110; and a modulated glucose concentration level 130. An output of controller 100 may include an insulin controller command 135. An output of controller 100 may include a glucagon controller command (not shown) for increasing an amount of glucose in the bloodstream as may be needed to avert hypoglycemic events.

According to a preferred embodiment of the invention, the insulin controller command 135 is perturbed with a perturbation signal 115, with the perturbed insulin signal instructing the pump (not shown) to release an amount of insulin corresponding to the perturbed insulin signal.

The patient's physiology 125 responds to the insulin received and actual food intake and exercise events 120, and develops a glucose level that is measured with a sensor 140. The output of sensor 140, due to physiological responses to perturbed insulin levels, therefore is understood as a perturbed glucose concentration level 130.

Consistent with the methods described above, controller 100 processes the perturbed glucose concentration signal 130 and discerns or updates the patient's medication sensitivity. Based on the patient's medication sensitivity, controller 100 adjusts the insulin controller command so as to tailor the pump command algorithm to more appropriately suit the patient's physiology.

The foregoing, non-exclusive methods enable ascertaining patient sensitivity to medication, including the time for responding, the extent of responsiveness to a given amount and the duration of responsiveness, that contour dosage algorithms and enable deployment of closed loop systems for medication infusion that permits users to live normal, healthy lives with minimal attention to pump maintenance.

While the preferred embodiment of the invention is intended to provide a closed-loop system requiring no attention for ordinary operation, another embodiment of the invention provides for aiding patients as to timing for undertaking activities that might cause great changes in glucose levels, such as exercising and ingesting a meal. Based on the method of discerning a patient's sensitivity to medication, in particular the lag time, described above, the invention responds to a patient's predicting or reporting of food/exercise and advises the patient as to when such activity would be best handled by the patient's physiology in view of the patient's sensitivity.

A number of embodiments have been described. Nevertheless, it will be understood that various modifications may be made. Accordingly, other embodiments are within the scope of the following claims.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

We claim:

1. Method of determining sensitivity by a patient to a medication delivered in a delivery amount at a delivery frequency, said method comprising:
perturbing the delivery amount by a perturbation amount at a perturbation frequency;
measuring a patient response to said perturbing; and
recovering an effect of said perturbing from the patient response and defining a sensitivity.

2. Method of claim 1 wherein the delivery amount and/or delivery frequency depends on the sensitivity.

3. Method of claim 1 wherein said perturbing and said recovering are configured according to principles of signal processing.

4. Method of claim 1 wherein said perturbing and said recovering are configured according to principles selected from single-tone perturbation; frequency key shifting; code division multiplexing; heterodyning; Wiener filtering; and combinations thereof.

5. Method of claim 1 wherein the perturbation frequency is less than the delivery frequency.

6. Method of claim 1 wherein said measuring has a measuring frequency that is less than the delivery frequency.

7. Method of claim 1 wherein the perturbation frequency maximizes a signal to noise ratio of the sensitivity.

8. Method of claim 1 wherein said recovering comprises demodulating the patient response.

9. Method of claim 1 wherein the perturbation frequency is variable.

10. Method of claim 1 wherein the perturbation frequency is selected to enable determination of a correspondence with the sensitivity.

11. Method of claim 1 wherein the perturbation amount is variable.

12. Method of claim 9 wherein the perturbation amount is variable.

13. Method of customizing a medication delivery according to claim 1 comprising controlling the delivery amount and/or the delivery frequency based on the sensitivity.

14. System for determining sensitivity by a patient to a medication delivered in a delivery amount at a delivery frequency, said system comprising:
a processor configured to be in communication with a pump and a sensor;
wherein:
the pump is responsive to said processor;
the sensor is configured to transmit a signal that corresponds to a physiological parameter;
said processor is configured to:
perturb the delivery amount by a perturbation amount at a perturbation frequency;
measure a patient response to said perturbing; and
recover an effect of the perturbation from the patient response and define a sensitivity.

15. System of claim 14 wherein said processor is configured to adjust the delivery amount and/or delivery frequency based on the sensitivity.

16. System of claim 14 wherein said processor is configured to perturb the delivery amount and recover the effect of the perturbation according to principles of signal processing.

17. System of claim 14 wherein said processor is configured to perturb the delivery amount and recover the effect of the perturbation according to principles selected from single-tone perturbation; frequency key shifting; code division multiplexing; heterodyning; Wiener filtering; and combinations thereof.

18. System of claim 14 wherein said processor is configured to maintain the perturbation frequency at less than the delivery frequency.

19. System of claim 14 wherein said processor is configured to measure at a measuring frequency that is less than the delivery frequency.

20. System of claim 14 wherein said processor is configured to maintain the perturbation frequency so as to maximize a signal to noise ratio of the sensitivity.

21. System of claim 14 wherein said processor is configured to demodulate the patient response.

22. System of claim 14 wherein said processor is configured to vary the perturbation frequency.

23. System of claim 14 wherein said processor is configured to maintain the perturbation frequency so as to enable determination of a correspondence with the sensitivity.

24. System of claim 14 wherein said processor is configured to vary the perturbation amount.

25. System of claim 22 wherein said processor is configured to vary the perturbation amount.
